(19) United States
(12) Patent Application Publication
(10) Pub. No.: US 2025/0387641 A1
(43) Pub. Date: Dec. 25, 2025

(54) In Situ Radiation Dosimetry

(71) Applicant: Wisconsin Alumni Research Foundation, Madison, WI (US)

(72) Inventors: Brian Pogue, Madison, WI (US); Xu Cao, Madison, WI (US)

(21) Appl. No.: 18/748,762

(22) Filed: Jun. 20, 2024

(51) Int. Cl. A61N 5/10

(52) U.S. Cl. CPC A61N 5/1028; A61N 5/1071; A61N 2005/1094

(57) ABSTRACT

A dosimeter for measurement of therapeutic radiation dose measures light absorption by body tissue caused by solvated electrons during treatment to produce in situ dose information for dose monitoring and therapy machine interlocking.

### STATEMENT REGARDING FEDERALLY SPONSORED RESEARCH OR DEVELOPMENT

--

### CROSS REFERENCE TO RELATED APPLICATION

--

### BACKGROUND OF THE INVENTION

The present invention relates to radiation dosimetry, the measurement of radiation dose used in medical radiation therapy and, in particular, to an in situ dosimetry technique measuring solvated electrons.

Radiation therapy (RT) employs high-energy, ionizing radiation to kill or control the growth of malignant tumors. Because such radiation can harm both healthy and malignant tissue proper control of dose is of great importance.

A recent development in radiotherapy is FLASH radiotherapy (FLASH-RT). Such techniques used extremely short pulses at ultrahigh dose rates (UHDR) more than 100 times those of conventional radiotherapy, an approach which appears to produce lower damage in normal tissues. This enhanced selectivity of FLASH-RT is thought to occur at average dose rates greater than 40 Gy/s per fraction. There is likely also a minimum dose per fraction required to see the benefit of FLASH; however because the models used to date to test this are variable, the exact dose threshold is not yet resolved.

While there is considerable interest in FLASH-RT, the ability to quantify the dose delivered in real time is poor. Common methods of dose measurement, including Gafchromic film or thermoluminescence detectors (TLD), are incapable of real time measurements. A commonly used dosimeter that provides real-time measurement, the ionization chamber, is highly dose-rate dependent and largely ineffective for short radiation pulses associated with FLASH-RT. Furthermore, in-situ radiation dose measurement directly at the site of delivery for Flash-RT is preferable. Implanted or intra-cavity dosimeters can provide highly accurate in-situ dose assessments. However, these methods involve a degree of invasiveness, which remains a critical consideration in their application.

The lack of a real time in-situ dosimeter for FLASH affects ongoing studies by admitting errors in measurement of the UHDR delivered dose which can lead to misinterpretation of the magnitude and value of the FLASH effect.

The lack of a real time in-situ dosimeter for FLASH-RT also makes it difficult to monitor the proper operation of the radiotherapy machine. Generally, such monitoring demands nano-to micro-second level sensing.

### SUMMARY OF THE INVENTION

The present invention provides a dosimeter that can make in vivo, real time measurements of radiation dose using a measure of solvated electrons. The measurement of solvated electrons directly in the tissue boosts the low signal strengths associated with solvated electrons measured in water chambers, for example, by 5-6 times, through a scattering in the tissue significantly increasing effective path length. Variations in tissue chemistry from known measurements in pure water may be accommodated through tissue characterization and comparison of absorption during radiation treatment to baseline absorption at other times. The invention is particularly useful for FLASH-type of radiation therapy because it promises a linear response to high-intensity radiation pulses of extremely short pulse durations.

More specifically, in one embodiment, the invention provides an in situ radiation dosimeter having an input for receiving a signal indicating a delivery of therapeutic radiation to a human body, a light source adapted to project light through the human body during the delivery of therapeutic radiation, and a light receiver adapted to receive light from the light source after passage through the human body to provide a light absorption measurement functionally dependent on solvated electrons in tissue of the human body that were produced by the radiation. An electronic computer executing a stored program or a custom electrical circuit receives the light absorption measurement and outputs a measure of radiation dose.

It is thus a feature of at least one embodiment of the invention to provide an improved understanding of dose in tissue. It is a further feature of at least one embodiment of the invention to greatly boost the signal strength of absorption measurements of solvated electrons by exploiting tissue scattering. It is a further feature of at least one embodiment of the invention to provide a dosimeter capable of operating with the short pulses and high intensity associated with FLASH radiotherapy.

The in situ radiation dosimeter may further include an appliance adapted to support the light source and light receiver against the human body for the communication of light therewith.

It is thus a feature of at least one embodiment of the invention to provide a practical system for in situ measurement that can be readily integrated with radiotherapy systems through an appliance holding and stabilizing the various components against the human body.

In one embodiment, the appliance may hold both the light source and light receiver facing a skin surface on one side of the body.

It is thus a feature of at least one embodiment of the invention to provide an appliance that can provide predetermined and fixed orientations and separations of the light sensors and light transmitters for consistent measurement of the body and/or for optical tomography.

The appliance may include a light shield opaque to light at the wavelength range positionable to block ambient light from receipt by detector.

It is thus a feature of at least one embodiment of the invention to reduce external light signals which may produce errors or inconsistencies in the measurement.

In one embodiment, the appliance may provide a probe sized to be inserted into a body cavity including either or both of a light source and light receiver positioned to face outward through the cavity when the probe is so inserted.

It is thus a feature of at least one embodiment of the invention to provide a system that can largely overcome the problem of variable air cavities and their effect on tissue dose measurements by stabilizing the cavity and locating the region of dose measurement removed from cavity effects.

In one embodiment, the appliance provides an adhesive patch for attaching either a light source or light cavity to the patient's skin.

It is thus a feature of at least one embodiment of the invention to provide complete flexibility in probe placement particularly useful for skin measurements associated with body cavity probes.

The light source and light receiver employ optical fibers having proximal ends proximate to the human body and distal ends passing into a container providing a Faraday shield and x-ray attenuating material equivalent to a 1.5 mm sheet of lead or more.

It is thus a feature of at least one embodiment of the invention to make use of the week absorption signal by reducing effects from high-dose radiation and incident x-ray scattering.

The light receiver may be one of multiple light receivers and the light transmitter may be one of multiple light transmitters and the dosimeter may include a multiplexer for using the multiple light transmitters and multiple light receivers to make multiple light absorption measurements functionally dependent on solvated electrons in tissue of the human body. The electronic computer may employ the multiple light absorption measurements to provide a tomographic reconstruction of dose.

It is thus a feature of at least one embodiment of the invention to provide increased spatial understanding of dose in tissue.

The electronic computer may provide an output based on a measure of dose exceeding a predetermined threshold and adapted to initiate a shutdown of the radiotherapy machine.

It is thus a feature of at least one embodiment of the invention to provide a high-speed dose measurement system suitable for shutdown of FLASH-type radiotherapy.

Generally, the computer may make two measurements of light transmission at predetermined times within 100 μs of the irradiation and is removed by at least 100 μs from the irradiation.

It is thus a feature of at least one embodiment of the invention to provide a baseline value through the tissue reducing the effects of variation in tissue type as may affect the measurement in tissue.

These particular objects and advantages may apply to only some embodiments falling within the claims and thus do not define the scope of the invention.

### BRIEF DESCRIPTION OF THE DRAWINGS

FIG. 1 is simplified perspective block diagram showing an apparatus for radiotherapy including a tomographic scanner and radiation therapy machine together with a patient supported on a patient table, the patient holding an appliance measuring the absorption light in patient tissue by solvated electrons, the apparatus further providing an electronic computer for processing data from the appliance according to a stored program and input from a user terminal;

FIG. 2 is a perspective view of the appliance of FIG. 1 having fiber optics attached to a multiplexer unit;

FIG. 3 is an elevational cross-section through the patient of FIG. 1 showing positioning of the appliance with respect to a region of interest;

FIG. 4 is a flowchart of the program of FIG. 1 showing the principal steps in implementing the present invention;

FIG. 5 is a simplified timing diagram showing pulses of a FLASH treatment and absorption measurement signals used by the present invention;

FIG. 6 is a figure similar to FIG. 2 showing an alternative implementation of the appliance, for example, for insertion into a body cavity;

FIG. 7 is a cross-sectional view similar to that of FIG. 6 showing a measurement region produced by the catheter implementation of FIG. 5 and surface-placed sensors or light sources;

FIG. 8 is an example screen display of an output of the present invention on the user terminal of FIG. 1 indicating tissue dose; and

FIG. 9 is a perspective view of a skin-mounted sensor in an alternative embodiment of the invention.

### DETAILED DESCRIPTION OF THE PREFERRED EMBODIMENT

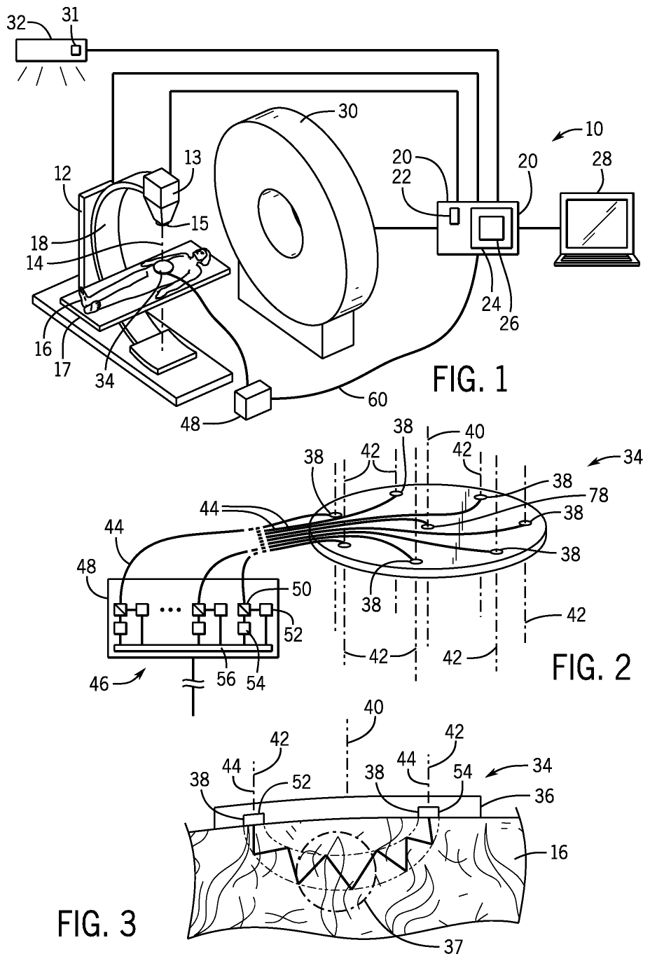

Referring now to FIG. 1, a radiotherapy system 10 suitable for practice of the present invention may provide a radiotherapy machine 12, for example, a Mobetron IORT FLASH radiotherapy system commercially available from IntraOp Medical Corporation of San Jose, California. This example radiotherapy machine 12 may employe an electron beam linear accelerator 13 providing a beam 14 with beam energies from 6 MeV to 12 MeV at a dose rate of 40 Gy/s to 400 Gy/s with pulse length variations from 0.5 μs 4 μs and repetition rate of 120 Hz.

The linear accelerator 13 may be supported on a motorized gantry 18 to control the angle of the beam 14 and its relative displacement with respect to a patient 16, the latter supported on a patient couch 17. The radiotherapy machine 12 may provide a flux sensor 15 allowing measurement of the flux of the beam 14.

The radiotherapy machine 12 may communicate with a controller computer 20, the latter providing for the control of the gantry 18 and linear accelerator 13 (either by providing or receiving timing pulses controlling or indicating operation of the beam 14), and may receive flux measurements from the flux sensor 15.

The controller computer 20 may provide one or more processors 22 communicating with electronic memory 24, the latter holding a stored program 26 executed by the processors 22 as will be described below. The controller computer 20 may also communicate with a user interface 28 for outputting data and receiving data from a user for control of the radiotherapy system 10 via the program 26.

A CT scanner 30 may operate in conjunction with the radiotherapy machine 12 to acquire tomographic images of the patient 16 used to identify a treatment region for radiotherapy, these tomographic images being registered with the position of the radiotherapy machine 12 to guide placement of the beam 14. The CT scanner 30 may also communicate with the controller computer 20 which may receive the tomographic images.

In one embodiment, the controller computer 20 may also communicate with a solid-state switch 31 controlling room lighting 32, for example, the latter implemented with light emitting diodes allowing for rapid extinguishing of illumination during operation of the beam 14 to be discussed below.

Referring now also to FIGS. 2 and 3, during treatment, the patient 16 may be fitted with an appliance 34 for monitoring radiation dose within the patient's tissue. In one embodiment, the appliance 34 may be a flexible mat 36, for example, of silicone or other flexible material adapted to be placed on and conform to the surface of the skin of the patient 16. The flexible mat 36 may be constructed of a material to provide dose buildup as is understood in the art and, for example, may be constructed of a water equivalent material approximating 5 to 10 mm of water in the path of the beam 14 to a proximate treatment region 37. Desirably the appliance 34 is opaque in the wavelength of light that will be used for monitoring dose by the present invention.

A set of sensor elements 38 are supported by the mat 36, for example, positioned in a circle about a central axis 40 generally aligned with the beam 14. In this embodiment, each of the sensor elements 38 may emit or receive light along respective axes 42 generally parallel to the axis 40 and directed into the patient 16 around the treatment region 37.

The sensor elements 38 may be discrete photodetectors and photo sensors but preferably are implemented by a distal ends of an optical fiber 44 communicating with a proximal end at a multiplexer unit 46 contained in a shielded enclosure 48. The shielded enclosure 48 may provide a Faraday shield against electromagnetic interference and an x-ray shielding equivalent to 1.0 mm of lead or more. The optical fibers 44 allows the multiplexer unit 46 to be placed away from patient-scattered x-rays and radio electromagnetic interference from the radiation therapy machine 12.

The multiplexer unit 46, in one embodiment, may include a set of beam splitters 50 associated with each optical fiber 44 of each sensor element 38 allowing alternately for the transmission of light from a light emitter 52 or the receipt of light by a light sensor 54 in the multiplexer unit 46 under the control of a data acquisition unit 56. The data acquisition unit 56 provides signals activating the light emitters 52 under the control of the controller computer 20 via communication cable 60 and receives signals for digital conversion from the light sensors 54 to be transmitted to the controller computer 20.

Each light emitter 52 provides for outputting of light including energy in an infrared absorption region subject to absorption by solvated electrons. This infrared absorption region is generally centered around the wavelength of 715 nm with a full width half maximum (FWHM) of approximately 600 nm or from 550-900 nm. The light sensors 54 will provide a similar wavelength sensitivity.

Light emitters 52 suitable for this use include but are not limited to lasers and light emitting diodes. Light sensors 54 suitable for this use include but are not limited to photomultiplier tubes and avalanche diodes including single photon avalanche diodes (SPADs). Importantly light sensor 54 provides microsecond and preferably nanosecond temporal resolution allowing measurements to be synchronized to the pulse duration of FLASH-RT. Improve signal-to-noise ratio measurements may be obtained by placing optical filters matched to the infrared absorption region on one or both of the light emitters 52 and light sensors 54.

During operation which will be described in more detail below, the multiplexer unit 46 may provide for a time division multiplexing of operation of the light emitters 52 and light sensors 54, for example, at a first time, providing illumination from one light emitter 52 while detecting received light from the remaining or a selected set of the sensor elements 38 acting as light sensors 54. At subsequent times, each of the different light emitters 52 is individually illuminated in round-robin fashion while the remaining sensor elements 38 operate as light sensors 54.

In an alternative mode of operation, the multiplexer unit 46 may provide for a frequency division multiplexing of light emitters 52, for example, at a first time, providing simultaneous illumination from multiple light emitters 52 limited to different subset wavelength regions of the infrared absorption region discussed above. These different emitted wavelengths are then simultaneously detected at multiple light sensors 54, for example, distinguishing among the wavelengths by filters and separate sensors, optical gratings, or interference techniques.

Referring to FIG. 3, in all cases, a portion of the light emitted from a light emitter 52 directed into the patient 16 will, through internal tissue scattering, follow a shallow arc to a receiving light sensor 54 opposed across the treatment region 37. Generally, the length of this arc will be limited by tissue absorption to around 10 cm and typically less than 20 cm; however, scattering provides an effective increase in the optical path length by 5 to 6 times. By cycling through the light emitter 52 and light sensors 54 a tomographic projections set of light attenuation measurements may be collected allowing optical tomographic reconstruction of the light absorption in the treatment region 37.

Alternatively, it will be appreciated that, in a simple embodiment, a single light emitter 52 and single light sensor 54 may be employed to provide a more generalized measurement of light absorption.

The light absorption will be a function of the presence of solvated electrons caused by the energy of the beam 14 interacting with the tissue of the treatment region 37 to provide a measure of deposited dose.

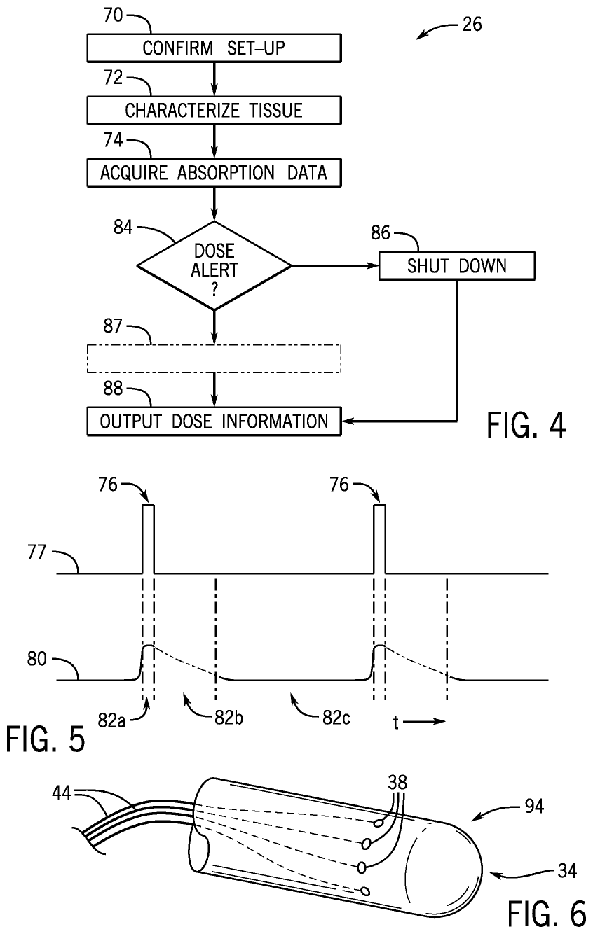

Referring now to FIGS. 1 and 4, the program 26 executing on the controller computer 20 may operate as indicated by process block 70 to selectively illuminate the light emitters 52 while sensing the light sensors 54 to confirm proper set up of the appliance 34 on the patient providing sufficient light signal between emitters 52 and sensors 54.

At process block 72 additional information characterizing the tissue and set up may be entered by the user through user interface 28 including, for example, the tissue type (e.g., an organ), patient information such as age or sex, and environmental factors, for example, temperature. This latter measurement may be obtained through a temperature probe in the appliance 34.

Tissue characterizing information and environmental information may be used to make empirical corrections to the relationship between light absorption and dose for different tissue types and conditions and may further include information obtained from the CT scanner 30, such as tissue x-ray attenuation, or derived from a measure of exit dose itself, for example, through an absorption spectrogram or the like.

At process block 74 absorption data is acquired either between a single pair of light emitter 52 and light sensor 54 or through frequency or time division multiplexing of multiple light emitters 52 and light sensors 54 for optical tomography as described above.

Referring now to FIG. 5, the acquisition of absorption data is coordinated with activation times 76 of the beam 14, being obtained either as an electrical signal 77 from the radiation therapy machine 12 or possibly by means of a stray x-ray sensor 78 (shown in FIG. 2), for example, attached to the appliance 34. While conventional radiotherapy uses the low dose rates on the order of 0.03 Gy/s, UHDR FLASH contemplated in the present invention uses dose rates of 40-400 Gy/s average or peak dose rates during activation times 76 of 105-106 in electron FLASH to reduce 0.1-10 Gy/pulse.

Using electrical signal 77 as a time reference, absorption signals 80 from the light sensors 54 can be partitioned into a first time 82a coincident with activation time 76 when electrons are being solvated, a second time 82b immediately following activation time 76 prior to recombination of the solvated electrons with reactive species from the water, and a third time 82c when the solvated electrons are largely fully recombined. The second time 82b will be on the order of 100 μs and will be typically much longer than time 82a which may, for example, be from 0.2 μs to 6 μs. Measurements taken during time 82c may be used to provide a baseline against which absorption is developed, indicated by time integrated values taken during either times 82a or 82b or a combination of these measures. A possible benefit to using values taken during time 82b is freedom from electrical or x-ray interference in the measurement process.

During time 82a, room lights may be extinguished by the controller computer 20 operating the switch 31 to reduce background light levels that could interfere with these measurements.

Referring still to FIG. 4, at decision block 84, initial dose calculations may be compared against expected dose levels (for example, as provided in programming to the radiation therapy machine 12 or representing a maximum safe dosage) and used to provide a machine shutdown per process block 86, turning off the radiotherapy machine 12 in the event of a malfunction that produces excessive dose. The ability to rapidly determine dose using the present invention makes this feasible. This decision block 84 may also consider the data from the flux sensor 15 if available.

If the dose is within acceptable ranges as determined at decision block 84, at optional process block 86, multiple dose measurements, for example, as part of an optical tomographic projections set may be reconstructed into a planar map of dose 90, for example, as shown in FIG. 8 and displayed as indicated by process block 88. Alternatively or in addition, numeric dose information 92 may be displayed, for example, dose rate, accumulated of dose, and the like. The length of time 82b may also provide insight into the micro chemical environment of the tissue and may be displayed. Additional measures that take into account the signal from the flux sensor 15 and devices to measure exit dose from the patient 16 may also be presented or combined to provide additional insight into the treatment. Details of the calculation of absorption are found in Mégrourèche J, et. als, Development of a hydrated electron dosimeter for radiotherapy applications: A proof of concept, Med Phys. 2023 November; 50(11): 7245-7251, doi: 10.1002/mp.16555. Epub 2023 Jun. 19, PMID: 37334736, hereby incorporated by reference in its entirety.

Referring now to FIG. 6, in an alternative embodiment, the appliance 34 may be designed for intracavity use, for example, as placed in the rectum, and in this regard provides a probe body designed to fill and hence stabilize the cavity during treatment. The appliance 34 again may support multiple sensor elements 38, for example, directed radially outward over a range of angles about an axis of the probe and cavity.

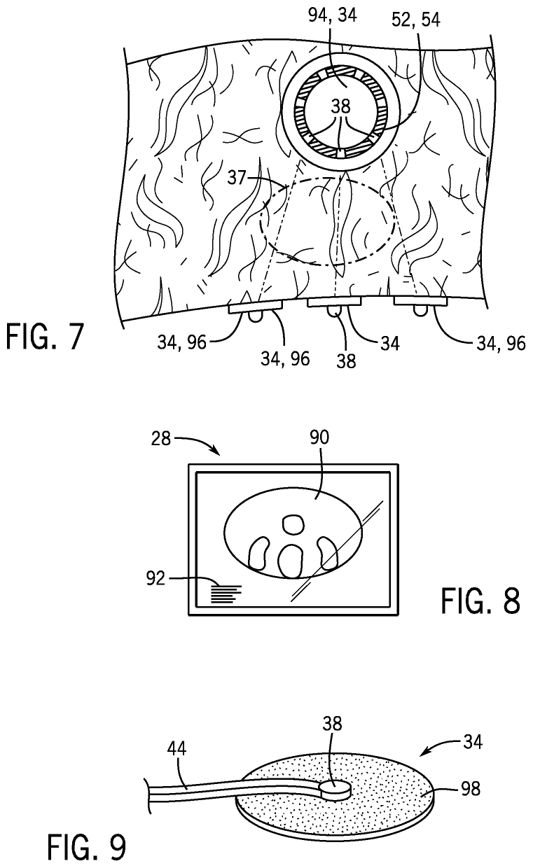

Referring to FIG. 7, for any given application, only a selected set of sensor elements 38 may be used, providing either light emitters 52 or light sensors 54 which will work in conjunction with surface appliances 96 having complementary light emitters 52 or light sensors 54. These surface appliances 96, for example, may be placed on the skin of the patient 16 proximate to the probe 94 when placed in the cavity to provide a light path that may determine dose in the cavity walls or nearby tissue.

Referring to FIG. 9, more generally sensor elements 38 may be supported by adhesive patches 98 to be applied on a case-by-case basis to particular regions of the skin allowing complete flexibility of placement and dose measurement radiotherapy.

While the invention has been discussed with respect to FLASH, it may be applicable to conventional radiotherapy providing the benefit of in-situ monitoring not previously available, for example, by integrating the low signals over a longer period of time and employing other signal processing techniques such as time-of-flight, diffuse optical tomography.

Certain terminology is used herein for purposes of reference only, and thus is not intended to be limiting. For example, terms such as “upper”, “lower”, “above”, and “below” refer to directions in the drawings to which reference is made. Terms such as “front”, “back”, “rear”, “bottom” and “side”, describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

When introducing elements or features of the present disclosure and the exemplary embodiments, the articles “a”, “an”, “the” and “said” are intended to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

References to “a computer” and “a processor” should be understood to include one or more devices that can communicate in a stand-alone and/or a distributed environment(s), and can thus be configured to communicate via wired or wireless communications with other processors, where such one or more processor can be configured to operate on one or more processor-controlled devices that can be similar or different devices. Furthermore, references to memory, unless otherwise specified, can include one or more processor-readable and accessible memory elements and/or components that can be internal to the processor-controlled device, external to the processor-controlled device, and can be accessed via a wired or wireless network. More generally, the term computer does not require a conventional von Neumann machine but should be considered to include electronic circuits that perform comparable functions using a stored program that may be implemented either in software or in interconnections between components. In this respect, a computer also may be considered to be a type of an electronic circuit.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications, are hereby incorporated herein by reference in their entireties

To aid the Patent Office and any readers of any patent issued on this application in interpreting the claims appended hereto, applicants wish to note that they do not intend any of the appended claims or claim elements to invoke 35 U.S.C. 112(f) unless the words “means for” or “step for” are explicitly used in the particular claim.

What we claim is:

1. An in situ radiation dosimeter comprising:
an input for receiving a signal indicating a delivery of therapeutic radiation to a human body;
a light source adapted to project light through the human body during the delivery of therapeutic radiation;
a light receiver adapted to receive light from the light source after passage through the human body to provide a light absorption measurement functionally dependent on solvated electrons in tissue of the human body; and
an electronic circuit operating to receive the light absorption measurement and to output a measure of radiation dose.

2. The in situ radiation dosimeter of claim 1 wherein the measure of dose is selected from the group consisting of total dose and dose rate.

3. The in situ radiation dosimeter of claim 1 further including an appliance adapted to support the light source and light receiver against the human body for a communication of light therewith.

4. The in situ radiation dosimeter of claim 3 wherein the appliance is adapted to hold both the light source and light receiver facing a skin surface on one side of the body.

5. The in situ radiation dosimeter of claim 4 where in the appliance further includes a light shield opaque to light at a wavelength range of absorption of solvated electrons positionable to block ambient light from receipt by the human body.

6. The in situ radiation dosimeter of claim 3 wherein the appliance provides a probe sized to be inserted into a body cavity including at least one of the light source and light receiver positioned to face outward through the body cavity when the probe is so inserted.

7. The in situ radiation dosimeter of claim 3 wherein the appliance provides an adhesive patch for attaching the at least one of the light source and light cavity to skin of the human body.

8. The in situ radiation dosimeter of claim 1 wherein the light source and light receiver employ optical fibers having proximal ends proximate to the human body and distal ends passing into a container providing a Faraday shield and x-ray attenuating material equivalent to a 1.5 mm sheet of lead or more.

9. The in situ radiation dosimeter of claim 1 wherein the light receiver is one of multiple light receivers and the light transmitter is one of multiple light transmitters and including a multiplexer for using the multiple light transmitters and multiple light receivers to make multiple light absorption measurements functionally dependent on sulfated electrons in tissue of the human body; and
wherein the electronic circuit employs the multiple light absorption measurements to provide a tomographic reconstruction of dose.

10. The in situ radiation dosimeter of claim 1 wherein the electronic circuit further provides an output based on a measure of dose exceeding a predetermined threshold adapted to initiate a shutdown of a radiotherapy machine providing the therapeutic radiation.

11. A radiotherapy machine comprising:
a patient support for holding a patient;
a source of radiation delivering pulses to the patient with dose rates in excess of 40 Gy/s; and
a dosimeter providing:
an input for receiving a signal indicating a delivery of pulse from the source of radiation;
a light source adapted to project light into the patient during the delivery of therapeutic radiation;
a light receiver adapted to receive light from the light source after passage through tissue of the patient to provide a light absorption measurement functionally dependent on solvated electrons in tissue of the patient; and
an electronic circuit executing a stored program to receive the light absorption measurement and to output a measure of radiation dose.

12. A method of radiation therapy comprising:
irradiating tissue of a patient with pulses of radiation having dose rates in excess of 40 Gy/s;
measuring light scattered through the tissue in a wavelength range of 550-900 nm at two predetermined times relative to the irradiating to determine a tissue attenuation of scattered light; and outputting a measure of dose by the irradiating of the tissue from the tissue attenuation at the two predetermined times.

13. The method of claim 12 wherein a first of the predetermined times is within 100 μs of the irradiation and a second of the predetermined times is removed by at least 100 μs from the irradiation.

14. The method of claim 12 wherein the measure of dose is selected from the group consisting of total dose and dose rate.

15. The method of claim 14 further including supporting the light source and light receiver on an appliance against the patient for a communication of light therewith; and
wherein the appliance is adapted to hold both the light source and light receiver facing a skin surface on one side of the patient.

16. The method of claim 14 further including supporting the light source and light receiver on an appliance against the patient for a communication of light therewith; and
wherein the appliance provides a probe sized to be inserted into a body cavity including at least one of the light source and light receiver positioned to face outward through the body cavity when the probe is so inserted.

17. The method of claim 14 further including supporting the light source and light receiver on an appliance against the patient for communication of light therewith; and
wherein the appliance provides an adhesive patch for attaching the at least one of the light source and light receiver to skin of the patient.

18. The method of claim 12 including covering the patient with a light shield opaque to light at the wavelength range positionable to block ambient light from receipt by the patient.

19. The method of claim 12 wherein the light receiver is one of multiple light receivers and the light transmitter is one of multiple light transmitters and may further include a multiplexer using the multiple light transmitters and multiple light receivers to make multiple light absorption measurements functionally dependent on solvated electrons in tissue of the patient; and wherein the output is a tomographic reconstruction of dose.

20. The method of claim 12 including comparing the measure of dose against a predetermined threshold adapted to initiate a shutdown of a radiotherapy machine when the measure of dose exceeds the predetermined threshold.
